(19) United States
(12) Patent Application Publication
(10) Pub. No.: US 2024/0282989 A1
(43) Pub. Date: Aug. 22, 2024

(54) PHENOTHIAZINE DERIVATIVES AND REDOX-FLOW BATTERIES

(71) Applicant: University of Kentucky Research Foundation, Lexington, KY (US)

(72) Inventors: Paban Sitaula, Morrisville, NC (US); Susan A. Odom, Lexington, KY (US)

(21) Appl. No.: 18/442,752

(22) Filed: Feb. 15, 2024

Related U.S. Application Data
(60) Provisional application No. 63/485,149, filed on Feb. 15, 2023.

(51) Int. Cl. H01M 8/04186; C07D 279/26; H01M 8/18

(52) U.S. Cl. CPC H01M 8/04186; C07D 279/26; H01M 8/188

(57) ABSTRACT

The presently-disclosed subject includes permanent-charge-bearing phenothiazinc derivatives that are redox-active, have beneficial solubility, and do not require supporting salts when used in redox flow battery applications.

### RELATED APPLICATIONS

This application claims priority from U.S. Provisional Application Ser. No. 63/485,149 filed Feb. 15, 2023, the entire disclosure of which is incorporated herein by this reference.

### GOVERNMENT INTEREST

This invention was made with government support under grant number 1805103 awarded by the National Science Foundation. The government has certain rights in the invention.

### TECHNICAL FIELD

The presently-disclosed subject matter generally relates to phenothiazine derivatives. In particular, certain embodiments of the presently-disclosed subject matter relate to permanent-charge-bearing phenothiazine derivatives that are redox-active, have beneficial solubility, and do not require supporting salts when used in redox flow battery applications.

### INTRODUCTION

A redox-flow battery (RFB) is a rechargeable electrochemical system consisting of two electrolyte reservoirs holding electrolytes for energy storage. One reservoir holds an electrolyte that is a posolyte and the other reservoir holds an electrolyte that is a negolyte.

The electrolytes are often redox active chemical species. The posolyte has a higher oxidation potential and is stored in a positive electrode containing reservoir, while the negolyte is stored in a negative electrode containing reservoir. These two electrolyte reservoirs are separated by an ion-selective nano-porous membrane separator, also referred to as an ion-exchange separator, which prevents the mixing of these two electrolytes.

The posolyte and negolyte are circulated with aid of an external pump. When an external potential is applied, the posolyte gets oxidized during charging and the negolyte gets reduced during discharging, completing one electrochemical cycle.

The electrodes used in the process can be made up of carbon fibers, felts, or papers, for example. An external chemical salt, sometimes referred to as a supporting salt, can be added to the electrolyte(s) to provide sufficient ionic conductivity. A supporting salt is often used when organic compounds and solvents are being employed, which generally have negligible conductivity. The ion-exchange separator is permeable to supporting salts, which is important for balancing electroneutrality. Meanwhile, most of redox-active materials used as electrolytes are not permeable, such that they are within their respective reservoirs, preventing the mixing of the posolyte and negolyte.

Redox flow batteries are useful grid-energy storage devises because power and energy densities can be independently scaled up. By tuning stereo-electronics of the compounds used as a redox-active material, the charging-discharging cycle can be varied, leading to a longer lifetime.

By storing compounds used as redox-active material are in distinct positive electrode (posolyte) and negative electrode (negolyte) reservoirs, there is a safety benefit. A vanadium-based RFB is the state-of-the-art RFB system, which employs vanadium ions as a redox-active material, and sulfuric acid as a supporting electrolyte. The largest vanadium based RFB is under commercialization by Rongke Co. Ltd., which is projected to be of 200 MW/800 MWh.

Vanadium-based RFBs suffer from low energy density, inhomogeneous active material distribution, low solubility of active materials, high viscosity of electrolyte solution, a need for use of a strong acid as a supporting electrolyte, and subjection to volatile pricing of vanadium metals, which has imposed challenges for the commercialization of vanadium RFB. Moreover, aqueous-based electrolytes have very narrow electrochemical window. Beyond a voltage of 1.5 V, water oxidizes to oxygen gas and reduces to hydrogen gas, both of which are flammable gases. Thus, it would be desirable to replace water with some organic solvents to achieved a prolonged electrochemical window (e.g., 4-5 V), which would lead to an increased cell voltage and energy density.

Phenothiazine is one of the most studied posolyte for RFB applications. By molecular engineering, it has been shown that this redox-active core can be tuned in various ways to increase its oxidation potential by incorporating a permanent charge bearing side chain, which can be used in organic batteries without supporting salts. However, the phenothiazine redox-active core is also known to have a single electron reversible redox event.

Accordingly, there remains a need in the art for improved phenothiazine derivatives that can be used in RFB applications.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes compounds that are phenothiazine derivatives. Some embodiments of the presently-disclosed subject matter include a compound selected from the following formulae:

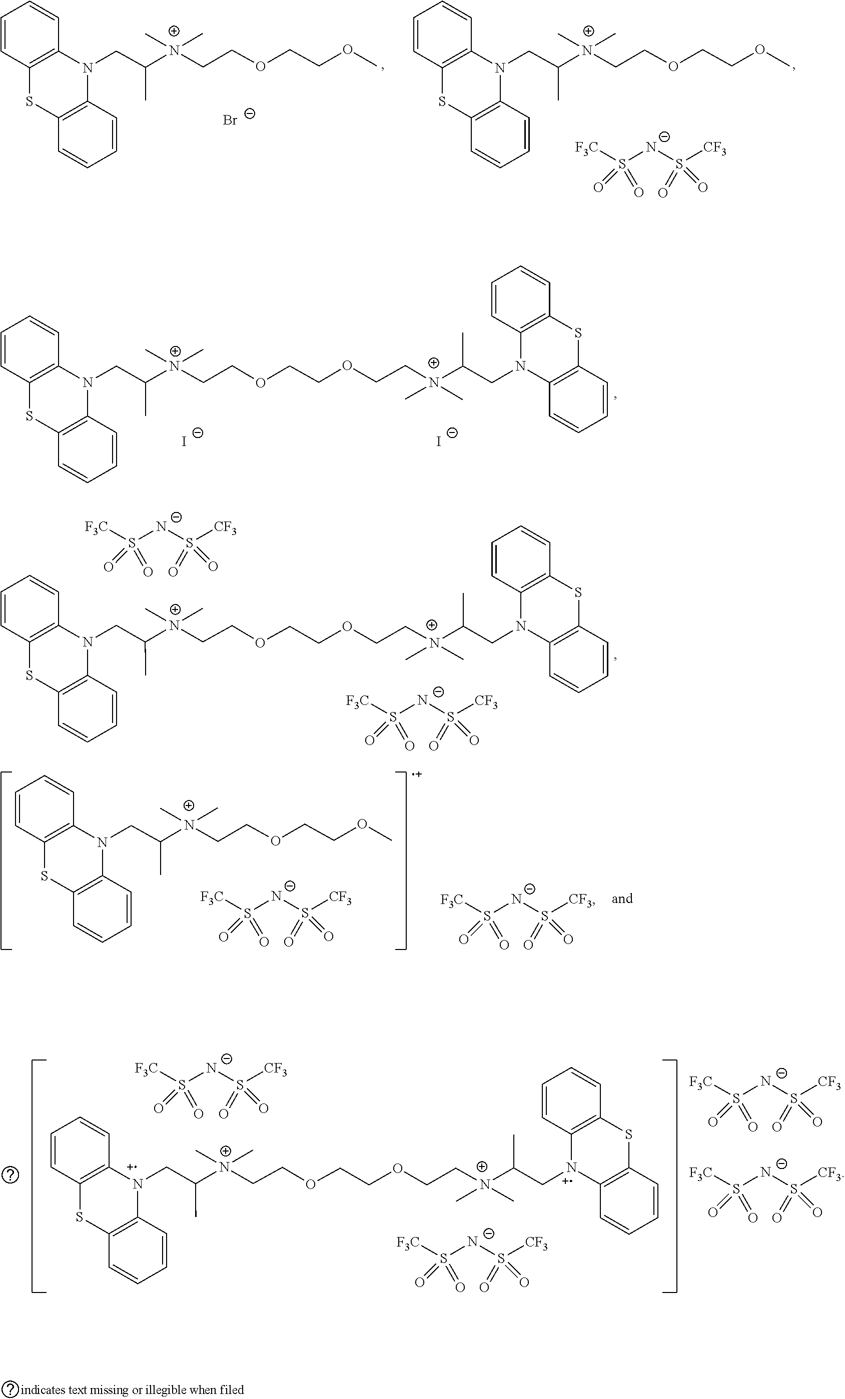

Some embodiments of the presently disclosed subject matter include a rechargeable battery comprising a negative electrode; a positive electrode; and an electrolyte comprising a compound as disclosed herein.

Some embodiments of the presently disclosed subject matter include a non-aqueous redox flow battery, comprising a negative electrode immersed in a first non-aqueous liquid electrolyte solution; a positive electrode immersed in a second non-aqueous liquid electrolyte solution, the second non-aqueous liquid electrolyte solution including at least one compound as disclosed herein; and a semi-permeable separator interposed between the negative and positive electrodes.

Some embodiments of the presently disclosed subject matter include an array comprising two or more of a battery as disclosed herein. In some embodiments, the two or more battery are connected in a series.

The presently-disclosed subject matter further includes a method of making a compound, as disclosed herein. The presently-disclosed subject matter further includes a method of making a battery, as disclosed herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A and 1B are spectra characterizing the synthesized monomer radical cation (FIG. 1A) and dimer radical cation (FIG. 1B).

FIGS. 2A and 2B are spectra from a stability study of 10 mM solution of monomer radical cation (FIG. 2A) and dimer radical cation (FIG. 2B) in acetonitrile.

FIGS. 3A-3C are cyclic voltammograms of 1 mM MEEPRT-TFSI in 0.1 M TEABF₄/ACN: Reversible 1^{st} oxidation (0.54 V, ipₐ/ip_{c}=1.06 with peak separation=59 mV, FIG. 3A), Full window (FIG. 3B), First oxidation scan (FIG. 3C).

FIGS. 4A-4C are cyclic voltammograms of 1 mM EEEPRT-TFSI in 0.1 M TEABF₄/ACN: Reversible 1^{st} oxidation (0.54 V, ipₐ/ip_{c}=1.06 with peak separation=59 mV, FIG. 4A), Full window (FIG. 4B), First oxidation scan (FIG. 4C).

FIGS. 5A-5C are overlaid cyclic voltammograms of 1 mM MEEPRT-TFSI and 1 mM EEEPRT-TFSI₂ in 0.1 M TEABF₄/ACN at scan rates of 10 mV/s (FIG. 4A), 100 mV/s (FIG. 4B), and 500 mV/s (FIG. 4C).

FIG. 6A-6D include results from a cross-over experimental set up with custom-built H-cell, including a plot showing cross-over fractions of redoxomers (FIG. 6A), and cyclic voltammetry of blank side of H-cell in various time for MEEPT (FIG. 6B), monomer (FIG. 6C), and dimer (FIG. 6D).

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes compounds that are phenothiazine derivatives. Some embodiments of the presently-disclosed subject matter include a compound selected from the following formulae:

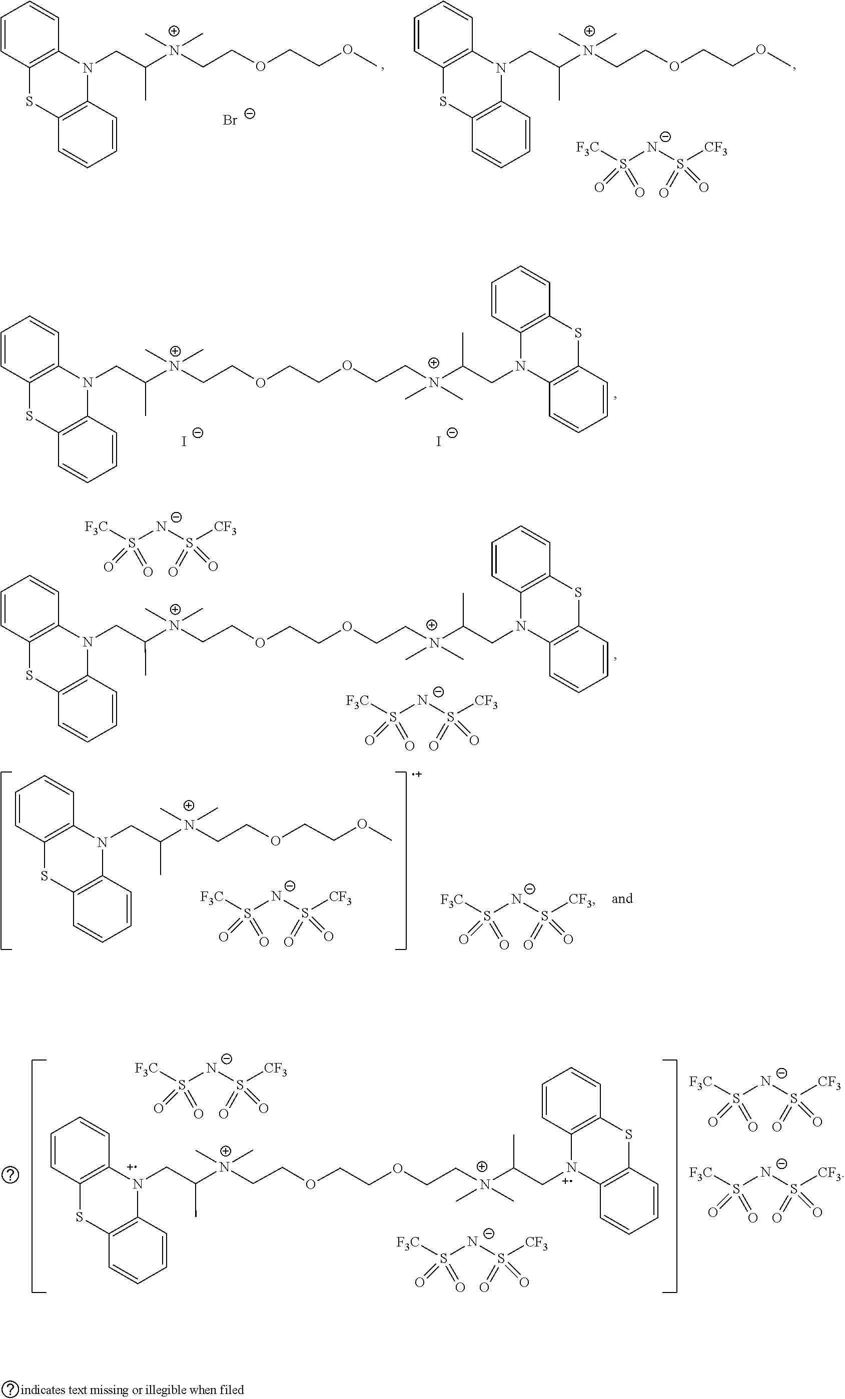

Some embodiments of the presently disclosed subject matter include a rechargeable battery comprising a negative electrode; a positive electrode; and an electrolyte comprising a compound as disclosed herein.

Some embodiments of the presently disclosed subject matter include a non-aqueous redox flow battery, comprising a negative electrode immersed in a first non-aqueous liquid electrolyte solution; a positive electrode immersed in a second non-aqueous liquid electrolyte solution, the second non-aqueous liquid electrolyte solution including at least one compound as disclosed herein; and a semi-permeable separator interposed between the negative and positive electrodes.

Some embodiments of the presently disclosed subject matter include an array comprising two or more of a battery as disclosed herein. In some embodiments, the two or more battery are connected in a series.

The presently-disclosed subject matter further includes a method of making a compound, as disclosed herein. The presently-disclosed subject matter further includes a method of making a battery, as disclosed herein.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

The term “electrolyte” is well understood to those of ordinary skill in the art and provides a charge-carrying pathway between the negative electrode and the positive electrode. The electrolyte can include a charge-carrying medium and a lithium salt. The electrolyte can also include a redox shuttle.

As used here, the term “redox shuttle” refers to an electrochemically reversible compound that can become oxidized at a positive electrode of a battery, migrate to a negative electrode of the battery, become reduced at the negative electrode to reform the unoxidized/less-oxidized shuttle species, and migrate back to the positive electrode. A redox shuttle can be an electroactive compound, which can be heterocyclic. A redox shuttle can protect against overcharging.

The term “negative electrode” is well understood to those of ordinary skill in the art and refers to one of a pair of electrodes that, under normal circumstances and when the battery/cell is fully charged, has the lowest potential. The negative electrode that can be used in connection with the presently-disclosed subject matter is not particularly limited and can be generally selected from those known in the art, for example, a graphitic anode.

The term “positive electrode” is well understood to those of ordinary skill in the art and refers to one of a pair of electrodes that, under typical circumstances, and when the battery/cell is fully charged, will have the highest potential that it can achieve under normal operation.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, in some embodiments±0.1%, in some embodiments±0.01%, and in some embodiments±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Synthesis of MEEPRT-Br

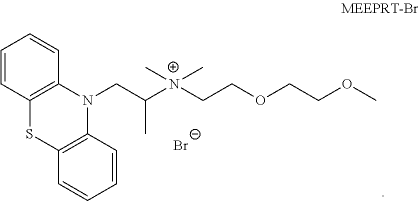 [SMILES: COCCOCC[N+](C)(C)C(C)CN1c2ccccc2Sc2ccccc21 ; [Br-]]

In a 250 mL oven dried round bottom flask fitted with a magnetic stirrer, promethazine (1.50 g, 5.29 mmol, 1 equiv.) was dissolved in anhydrous THF (30 mL) under nitrogen. After complete dissolution, 1-(2-bromoethoxy)-2-(2-methoxyethoxy)ethane (MEEBr) (1.56 g, 6.88 mmol, 1.3 equiv.) was added into the flask. The flask was heated to reflux in a reflux set up for 48 h after which solvents were evaporated to dryness in a rotary evaporator. The oil obtained as a crude product was washed with cold ether four times and the supernatant liquid was poured off to get MEEPRT-Br product (2.32 g, 4.97 mmol, 94% yield) as a spongy white solid.

### Example 2: Synthesis of MEEPRT-TFSI (Monomer)

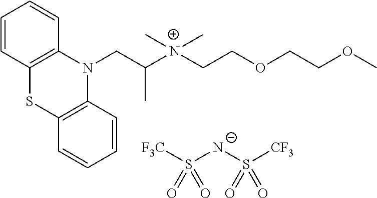 [SMILES: COCCOCC[N+](C)(C)C(C)CN1c2ccccc2Sc2ccccc21 ; O=S(=O)([N-]S(=O)(=O)C(F)(F)F)C(F)(F)F]

In a 250 mL oven dried round bottom flask fitted with a magnetic stirrer, MEEPRT-Br (2.0 g, 4.28 mmol, 1 equiv.) was dissolved in distilled water (30 mL) and was bubbled with nitrogen gas for 15 min. After that, lithium bis(trifluoromethanesulfonyl)imide (2.46 g, 8.56 mmol, 2 equiv.) was added into the flask under nitrogen gas and was stirred for 24 h, after which solvents were evaporated to dryness in a rotary evaporator. The crude product was washed with ether four times and the supernatant liquid was poured off to get MEEPRT-TFSI monomer (2.63 g, 3.94 mmol, 91% yield) as a clear viscous liquid which was dried in a vacuum oven at 30° C. 1H NMR (400 MHZ, DMSO-d6) δ 7.23-7.21 (m, 6H), 7.06 (t, J=7.38 Hz, 2H), 4.6-4.6 (dd, J=14.49, 4.11 Hz, 2H), 4.2-4.1 (m, 3H), 3.9 (m, 1H), 3.8-3.7 (m, 4H), 3.70-3.62 (m, 4H), 3.60-3.52 (m, 6H), 3.20 (s, 3H) ppm. ¹³C NMR (400 MHZ, DMSO-d6) δ 144.3, 127.9, 27.7, 125.3, 123.6, 119.5 (q, J=1.27 Hz), 116.7, 69.1, 66.8, 63.9, 61.1, 48.8, 45. 8, 12.2 ppm. 19F NMR (400 MHZ, DMSO-d6) δ −78.3 ppm. Anal. calcd. for C₂₄H₃₁F₆N₃O₆S₃; C, 43.17; H, 4.68; N, 6.29. Found C, 43.21; H, 4.68; N, 6.74.

### Example 3: Synthesis of EEEPRT-I Dimer

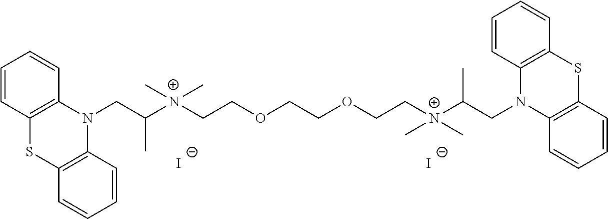 [SMILES: CC(CN1c2ccccc2Sc2ccccc21)[N+](C)(C)CCOCCOCC[N+](C)(C)C(C)CN1c2ccccc2Sc2ccccc21 ; [I-] ; [I-]]

In an oven-dried 50 mL round-bottom flask equipped with a magnetic stirrer, promethazine (1.50 g, 5.29 mmol) and bis(iodoethoxy)ethane (0.65 g, 1.76 mmol), and anhydrous THF (15 mL) were combined under nitrogen atmosphere. The flask was then connected to a reflux condenser and refluxed for 72 h in the dark, after which it was cooled down to room temperature and the THF was evaporated using a rotary-evaporator. The foamy-solid crude product was rigorously washed with boiling ethyl acetate (10 mL×20 times) to isolate a white amorphous solid as the pure product (1.26 g, 76% yield). ¹H NMR (400 MHZ, DMSO-d6) δ 7.33-7.19 (m, 12H), 7.16-6.95 (m, 4H), 4.66 (dd, J=14.45, 4.06 Hz, 2H), 4.11 (dd, J=14.45, 8.44 Hz, 2H), 3.90-3.65 (m, 8H), 3.65-3.54 (m, 2H), 3.52-3.29 (m, 4H), 3.15 (d, J=9.51 Hz, 12H), 1.39 (d, J=6.38 Hz, 6H) ppm. ¹³C NMR (100 MHZ, DMSO-d6) δ 144.2, 128.0, 127.7, 125.2, 123.5, 116.8, 69.1, 66.9, 61.12, 50.8, 48.9, 48.7, 45.9, 12.5 ppm.

### Example 4: Synthesis of EEEPRT-TFSI₂ (Dimer)

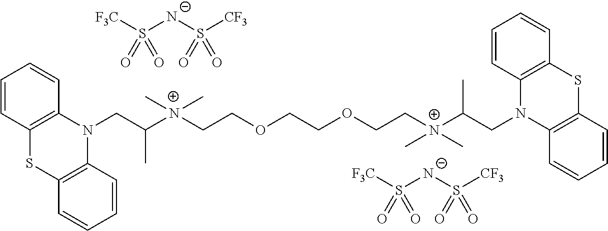 [SMILES: CC(CN1c2ccccc2Sc2ccccc21)[N+](C)(C)CCOCCOCC[N+](C)(C)C(C)CN1c2ccccc2Sc2ccccc21 ; O=S(=O)([N-]S(=O)(=O)C(F)(F)F)C(F)(F)F ; O=S(=O)([N-]S(=O)(=O)C(F)(F)F)C(F)(F)F]

In an oven-dried 100 mL round-bottom flask equipped with a magnetic stirrer, MEEPRT-I dimer (3.30 g, 3.52 mmol) was added to 25 mL of distilled water. The resulting mixture was heated to 85° C. for 10 minutes. To this slightly turbid reaction mixture, lithium bis(trifluoromethanesulfonyl)imide (3.03 g, 10.56 mmol, 3 equiv.), was added in portions. The reaction was heated at 85° C. for 12 h after which it was cooled to room temperature. The residue was washed with water and then diethyl ether. The white solid was dried in vacuum oven at 33° C. overnight to get pure product (4.03 g, 92% yield). 1H NMR (400 MHZ, DMSO-d6) δ 7.23-7.21 (m, 12H), 7.06 (t, J=7.38 Hz, 4H), 4.66-4.61 (dd, J=14.51, 4.13 Hz, 2H), 4.19-4.09 (m, 2H), 3.89-3.85 (m, 2H), 3.80-3.73 (m, 4H), 3.70-3.62 (m, 2H), 3.60-3.52 (m, 2H), 3.46-3.34 (m, 4H), 3.12 (d, J=9.1 Hz, 12H), 1.39 (d, J=6.5 Hz, 6H) ppm. ¹³C NMR (400 MHZ, DMSO-d6) δ 144.3, 127.9, 27.7, 125.3, 123.6, 119.5 (q, J=1.27 Hz), 116.7, 69.1, 66.8, 63.9, 61.1, 48.8, 48.5, 45. 8, 12.4 ppm. 19F NMR (400 MHZ, DMSO-d6) δ −78.6 ppm. Anal. calcd. for C44H52F12N6O10S6; C, 42.79; H, 4.22; N, 6.77. Found C, 42.44; H, 4.21; N, 6.75.

### Example 5: Synthesis of MEEPRT-(TFSI)2 Monomer Radical Cation

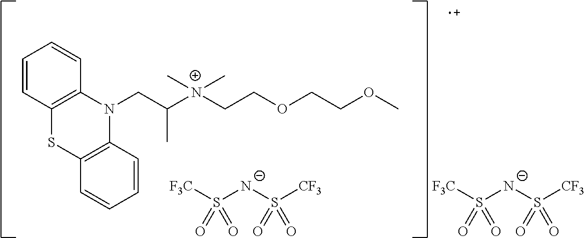

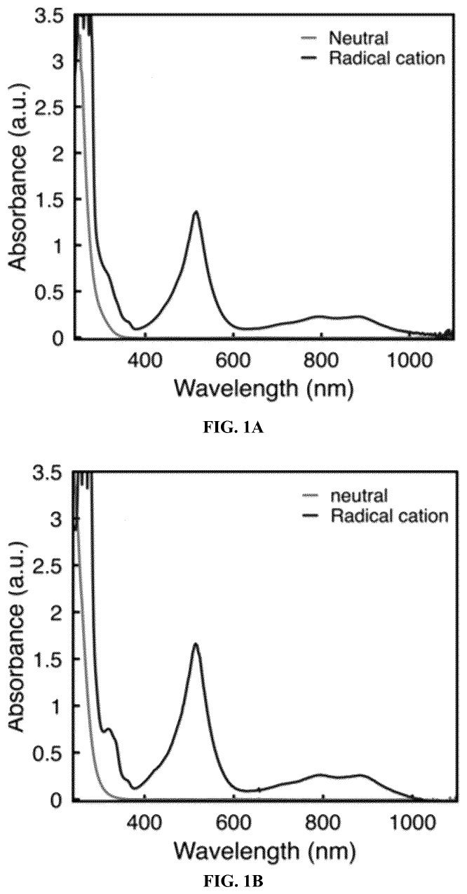

In an oven-dried round bottom flask fitted with a magnetic stirrer, MEEPRT-TFSI (1.0 g, 1.50 mmol, 1 equiv.) was dissolved in anhydrous 15 mL of distilled water and the mixture was bubbled with nitrogen for 10 min after which silver bis(trifluoromethanesulfonyl)imide (0.70 g, 1.80 mmol, 1.2 equiv.) was added under nitrogen environment. The color of the reaction mixture turned deep red within few seconds. The generation of the radical cation form was confirmed with UV-vis spectroscopy evidenced with intense absorption peak around 570 nm wavelength (FIG. 1A). After 10 min, the reaction was quenched with anhydrous ether. Thus, obtained precipitates were collected by filtering through a sintered glass funnel. the residue was washed with dry ether three times to wash away any remaining starting materials under nitrogen blanket to afford 1.21 g, (85% yield) of radical cation as a deep red solid.

### Example 6: Synthesis of EEEPRT-(TFSI)4 Dimer Radical Cation

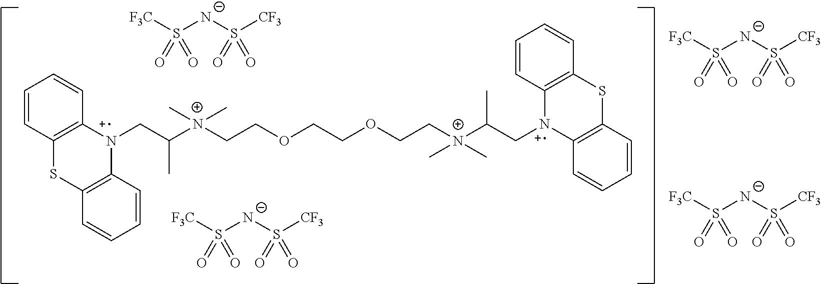

In an oven-dried round bottom flask fitted with a magnetic stirrer, MEEPRT-TFSI2 dimer (1.0 g, 0.80 mmol, 1 equiv.), was dissolved in anhydrous 20 mL distilled water (degassed by bubbling nitrogen gas for 15 min) by heating the reaction mixture to 85° C. Silver bis(trifluoromethanesulfonyl)imide (0.75 g, 1.92 mmol, 2.4 equiv.) was added under nitrogen environment. The color of the reaction mixture turned deep red within few seconds. The generation of the radical cation form was confirmed with UV-vis spectroscopy evidenced with intense absorption peak around 570 nm wavelength (FIG. 1B). After 10 min, the reaction was quenched with anhydrous ether. Thus, obtained precipitates were collected by filtering through a sintered glass funnel. the residue was washed with dry ether 3 times to wash away any remaining starting materials under nitrogen blanket to afford 1.18 g (82% yield) of radical cation as a deep red solid.

### Example 7: Solubility Measurement

The MEEPRT-TFSI monomer is a viscous liquid at room temperature, the miscibility was determined in acetonitrile and 0.1 M TEATFSI in acetonitrile. In both solvents, it is miscible when mixed with 1:10, 10:1, and 1:1 ratio by wt. of acetonitrile:redoxomers. Solubility of redoxomers were determined by NMR method using 1,4-bis(trifluoromethyl)benzene as an internal standard. The solubility of dimer-TFSI salt is found to be 1.6 M in acetonitrile and 1.2 M in 0.1 M TEATFSI in acetonitrile.

The miscibility of monomer and dimer radical cations in acetonitrile were studied by mixing acetonitrile and radical cations in the ratio of 1:1, 10:1 and 1:10 by wt., and they were found completely miscible in all ratio of acetonitrile and radical cations.

### Example 8: Stability Study of the Radical Cations

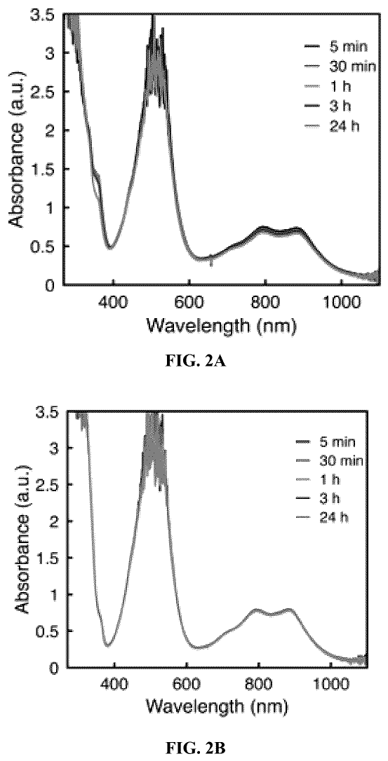

The stability of radical cations was studied by UV-vis spectroscopy using 10 mM solution of radical cations in in acetonitrile (FIG. 2). The solution was analyzed over 24 h. There was no significant loss of absorption intensity in 24 h.

### Example 9: Cyclic Voltammetry of Monomer and Dimer

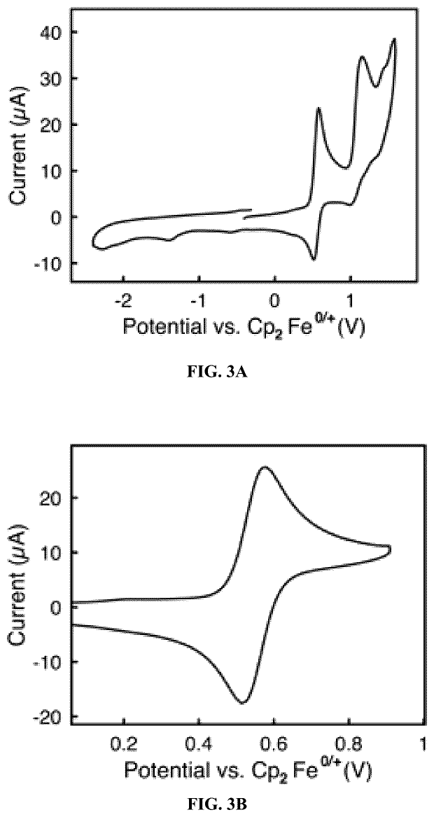

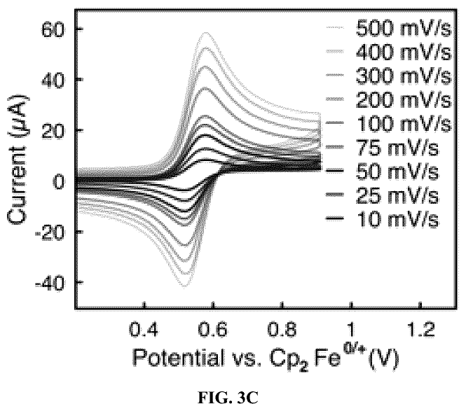

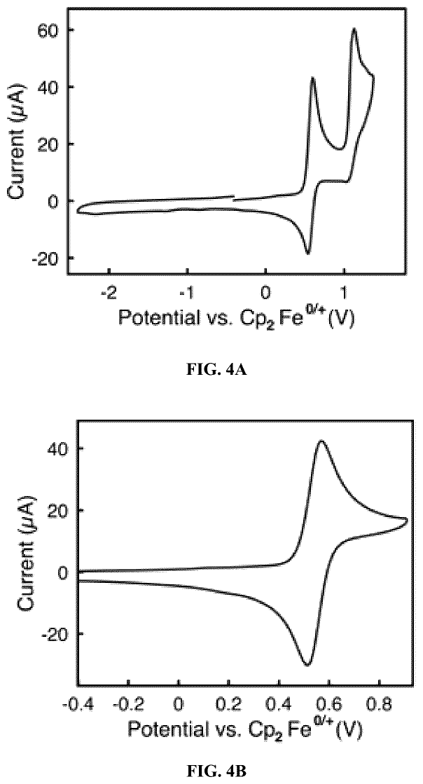

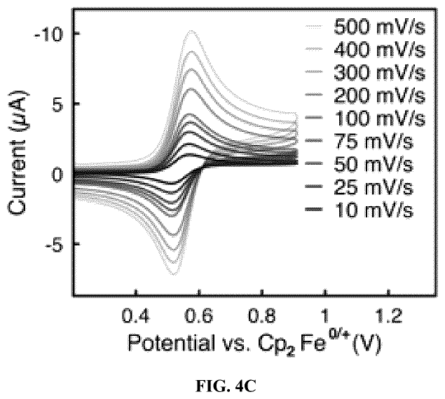

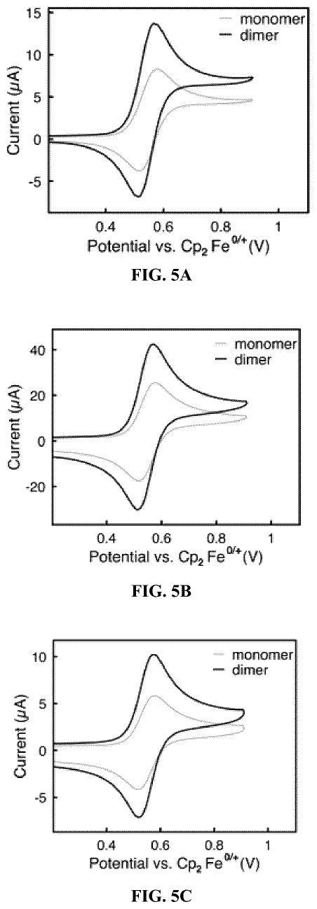

The electrochemical stability and reversibility of redox-events were determined by cyclic voltammetry experiments, which were carried out in a CH Instrument potentiostat with glassy carbon electrode as a working electrode, Pt-wire electrode as a counter electrode and a silver electrode as a reference electrode. The experiments were carried out in a argon-filled glovebox taking a 10 mM solution of newly synthesized monomer and dimer redoxomers. The monomer involves a single electron reversible redox reaction with oxidation potential of 0.54 V referenced to Fc/Fc⁺, while dimer undergoes a two-electron reversible redox event at the same potential. The two-electron redox-event was determined by overlapping the current vs potential curve of monomer and dimer, which showed almost double current intensity at all scan rate (FIGS. 3-5).

### Example 10: Cross-Over Experiments

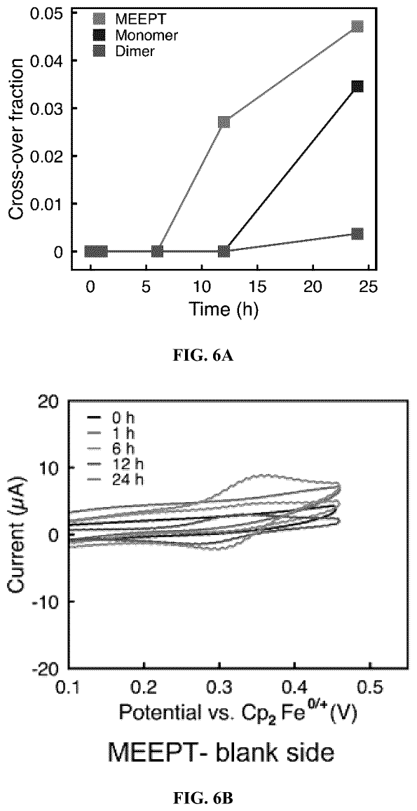

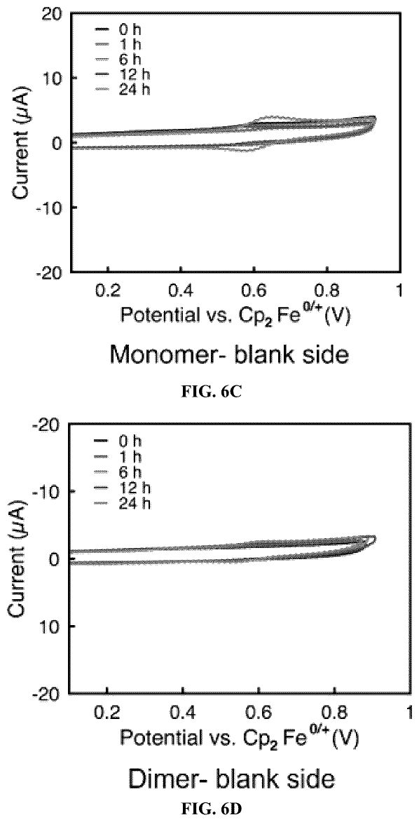

Cross-over of the active materials from anodic chamber to cathodic chamber and vice-versa is the major parameter contributing capacity drop and hence the lifetime of the redox-flow batteries. We compared cross-over fractions of our newly synthesized redoxomers with our previous redoxomers such as MEEPT and EPRT-TFSI using a custom-built H-cell. A stack of two layer of FAPQ-375-PP membrane pre-soaked in acetonitrile for 24 h was used as membrane between two compartments of H-cell. For each set of experiment, a 0.1 M solution of redoxomer in anhydrous acetonitrile on the left side and acetonitrile (blank solution) on the right side. The cross over fractions were determined by cyclic voltammetry of the solution from the blank side in 0 h, 2 h, 4 h, 8 h, 12 h, and 24 h in a argon-filled glovebox. The cross over fractions for MEEPT, EPRT-TFSI (compared to data from reference . . . ), monomer and dimer were found to be 0.0471, 0.0420, 0.0346, and 0.0037, respectively. This shows that the monomer has significant improvement in cross-over compared to MEEPT and little improvement compared to EPRT-TFSI, while dimer has much improvement in cross-over rate compared to all other three redoxomers (FIG. 6A-D).

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Yan, Yichao, Paban Sitaula, Susan A. Odom, and Thomas P. Vaid (2022) “High Energy Density, Asymmetric, Nonaqueous Redox Flow Batteries without a Supporting Electrolyte,” ACS Appl. Matter. Interfaces. doi.org/10.1021/acsami.2c10072
2. U.S. Pat. No. 10,854,911, Susan A. Odom, et al. “1,9,10-Substituted Phenathiazine Derivatives With Strained Radical Cations And Use Thereof,” issued Dec. 1, 2020.
3. U.S. Pat. No. 10,954,201, Susan A. Odom, et al. “Two-Electron Donating Phenothiazine and Use Thereof,” issued Mar. 23, 2021.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A compound selected from the group consisting of:

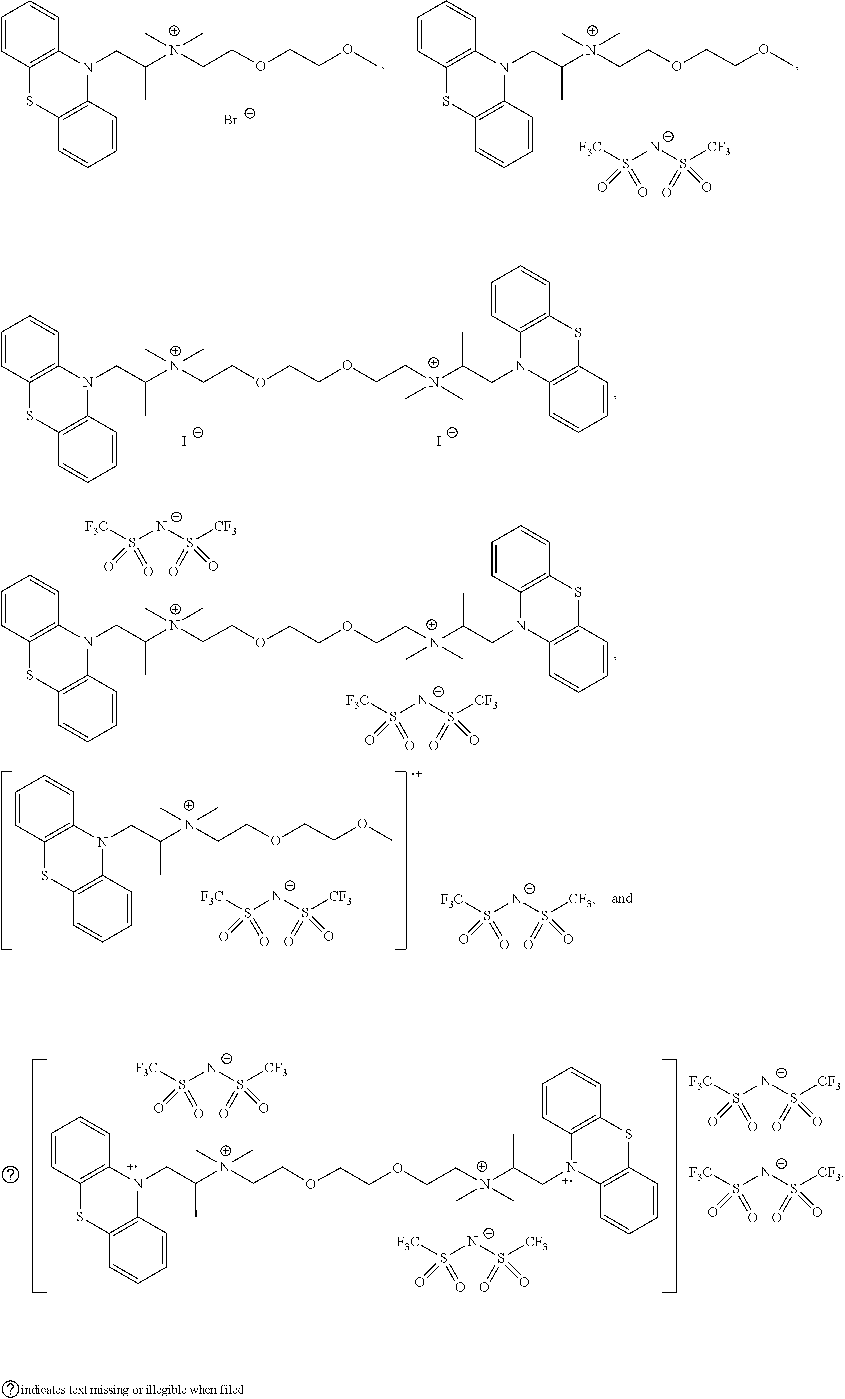

2. A rechargeable battery comprising:
a negative electrode;
a positive electrode; and
an electrolyte comprising the compound of claim 1.

3. A non-aqueous redox flow battery, comprising:
a negative electrode immersed in a first non-aqueous liquid electrolyte solution;
a positive electrode immersed in a second non-aqueous liquid electrolyte solution, the second non-aqueous liquid electrolyte solution including at least one compound according to claim 1; and
a semi-permeable separator interposed between the negative and positive electrodes.

4. An array comprising two or more of the battery of claim 3.

5. The array of claim 8, wherein the two or more battery are connected in a series.
